(19) United States
(12) Patent Application Publication
(10) Pub. No.: US 2012/0321682 A1
(43) Pub. Date: Dec. 20, 2012

(54) COSMETIC COMPOSITIONS COMPRISING MESOPOROUS TITANIA AND METHODS OF USE

(75) Inventor: Gloria Alejandra Camacho Bragado, Chicago, IL (US)

(73) Assignee: L'OREAL, Paris (FR)

(21) Appl. No.: 13/578,743

(22) PCT Filed: Feb. 15, 2011
(86) PCT No.: PCT/US11/24861

§ 371 Date: Aug. 13, 2012

Related U.S. Application Data
(60) Provisional application No. 61/306,245, filed on Feb. 19, 2010.

(51) Int. Cl. A61K 8/29; A61K 8/368; A61Q 5/12; A61Q 1/12; A61Q 1/06; A61Q 5/02; A61K 8/02; A61Q 17/04

(52) U.S. Cl. 424/401; 424/59; 424/60

(57) ABSTRACT

The disclosure relates to a cosmetic composition comprising mesoporous titania with a particle size optimized to provide UV light absorption without the risk of penetrating the skin; and to methods for preventing or reducing ultraviolet radiation damage to the body. The particle size of the mesoporous titania is typically from about 0.3 microns to about 250 microns. The pore size of the mesoporous titania is typically from about 2 nm to about 100 nm. The crystallite/wall size of the mesoporous titania is typically from about 2 nm to about 200 nm.

TECHNICAL FIELD

The disclosure relates to cosmetic compositions comprising mesoporous titania and a dermatologically acceptable carrier; and to methods of using the cosmetic compositions for preventing or reducing ultraviolet light damage to the body.

### BACKGROUND OF THE DISCLOSURE

The negative effects of exposure to ultraviolet (“UV”) light are well known. Prolonged exposure to sunlight causes short term damage such as sunburn and premature aging of the skin and can dry and damage hair. When skin is exposed to UV light having a wavelength of from about 290 nm to about 400 nm, long term damage can lead to serious conditions such as skin cancer. As a result of such health hazards caused by excessive sun exposure, sunscreen products have been developed.

The market provides a variety of sunscreen products. Consumers desire sunscreen products that effectively shield against the sun's rays and are aesthetically pleasing upon application to the skin. Sunscreen products can employ inorganic compounds such as titanium dioxide that serve as UV filters or physical sun blocking agents, thereby minimizing or preventing the exposure of the skin to UV radiation. Unfortunately, many commercial sunscreen products that contain inorganic UV filters produce an aesthetically undesirable white residue or tint when applied onto skin. Therefore, there exists a need for products containing inorganic UV filters that provide adequate, if not better UV protection to the skin and have an aesthetically pleasing appearance upon application.

### BRIEF SUMMARY OF THE DISCLOSURE

The disclosure relates to cosmetic compositions for providing UV light absorption comprising mesoporous titania and a dermatologically acceptable carrier; and to methods for preventing or reducing UV radiation damage to the body using the cosmetic compositions. The inventors discovered that mesoporous titania in cosmetic compositions can block harmful rays while appearing clear to the human eye. Titania and other metal oxides are very effective at blocking UV light. Furthermore, they are among the only chemicals capable of shielding both ultraviolet A (“UVA”) (skin-wrinkling) and ultraviolet B (“UVB”) (cancer-causing) rays. Accordingly, the present disclosure relates to the use of mesoporous titania in ultra-violet light absorbing cosmetic compositions for protecting the skin from aging and to fend off potentially deadly skin cancer. Due to its particle and pore size, the mesoporous titania provides optimal UV protection and at the same time, can be formulated into a composition that does not impart an undesireable white appearance to the skin upon application. Furthermore, the compositions are safe for application to the skin.

The cosmetic compositions comprise mesoporous titania and typically absorb UVA light from about 320 nm to about 400 nm and/or UVB light from 280 nm to 320 nm The particle size of the mesoporous titania can vary but will typically be from about 0.3 microns to about 300 microns, from about 0.3 microns to about 150 microns, or from about 0.5 microns to about 50 microns. The pore size of the mesoporous titania will vary but is typically from about 2 nm to about 100 nm or from about 2 nm to about 50 nm. The crystallite/wall size of the mesoporous titania will also vary but is typically from about 2 nm to about 200 nm, from about 2 nm to about 150 nm, or from about 5 nm to about 100 nm.

Generally, the cosmetic composition will comprise mesoporous titania from about 0.5% to about 75%, from about 1% to about 50%, or from about 1% to about 25% by weight of the total weight of the composition. The exact amount will vary depending upon the desired Sun Protection Factor (“SPF”) and the combination of other ingredients included in the cosmetic composition. SPF is a commonly used measure of photoprotection of a material against erythema. See, e.g., Federal Register, Vol. 43, No. 166, pp. 38206-38269, Aug. 25, 1978.

The cosmetic compositions may optionally include an additional ingredient capable of absorbing UV light. Among additional ingredients that can be employed in the cosmetic compositions are avobenzone, cinnamic acid derivatives (such as octylmethoxy cinnamate), octyl salicylate, oxybenzone, non-mesoporous titania, zinc oxide, or any mixtures thereof. In one embodiment the additional substance is an organic ingredient selected from the group consisting of octinoxate, octisalate, homosalate, octocrylene, para-aminobenzoic acid, cinoxate, dioxybenzone, methyl anthranilate, octocrylene, padimate O, ensulizole, sulisobenzone, trolamine salicylate, ecamsule; and mixtures thereof. The additional substances may be present from about 0.5% to about 7% , about 1% to about 50%, or from about 1% to 25 weight % of the total weight of the composition.

The cosmetic compositions can be in the form of a protective care composition for the skin including the face, the neck, the hands, the feet, or other areas of the body such as the hair and nails. Non-limiting examples include day creams or lotions, night creams or lotions, moisturizer, salves, sunscreens, oils, ointments, gels, body milks, makeup (e.g., a foundation or a lipstick), artificial tanning compositions, emulsifiers, or a solid which is poured or cast as a stick or a dish, for example. The mesoporous materials are ideal for use in a foundation product because they can achieve high camouflage and blurring effects to result in the perception of a natural appearance. In one embodiment, the cosmetic compositions are formulated as a sunscreen, lotion, cream, gel, spray, powder, foundation, lipstick, shampoo, and/or conditioner.

### DETAILED DESCRIPTION OF THE DISCLOSURE

The term “comprising” (and its grammatical variations) as used herein is used in the inclusive sense of “having” or “including” and not in the exclusive sense of “consisting only of” The terms “a” and “the” as used herein are understood to encompass the plural as well as the singular. Other than in the operating examples, or where otherwise indicated, all numbers expressing quantities of ingredients and/or reaction conditions are to be understood as being modified in all instances by the term “about,” meaning within 10% to 15% of the indicated number.

The term “titania” refers to titanium dioxide (TiO₂), also known as titanium(IV) oxide.

The term “mesoporous” refers to a material that is formed by sub-micron-sized particles (referred as crystallites in this document) that are held together by strong covalent forces forming micron-sized aggregates (referred as “particles” in this document) and leaving interparticle spaces (referred as pores).

The cosmetic compositions of the instant disclosure provide UV light absorption in a range from about 280 nm to about 400 nm, which is accomplished, at least in part, by the inclusion of photoactive mesoporous titania. Mesoporous titania provides a network of sub-micron-sized domains of crystalline titania that is thermally and mechanically stable (no or very little particle detachment or decomposition). It also presents high absorbance in various UV ranges of interest for cosmetic compositions including the UVB region responsible for most of the melanoma and other radiation-induced skin diseases.

The particle size of the mesoporous titania can vary but will typically be from about 0.3 microns to about 300 microns, from about 0.3 microns to about 150 microns, or from about 0.5 microns to about 50 microns. The pore size of the mesoporous titania will vary but is typically from about 2 nm to about 100 nm or from about 2 nm to about 50 nm. The crystallite/wall size of the mesoporous titania will also vary but is typically from about 2 nm to about 200 nm, from about 2 nm to about 150 mil, or from about 5 nm to about 100 nm.

Various methods of producing photoactive mesoporous titania exist and particular methods allow for the manipulation of the morphology and properties of the resulting product. Wu et al., J. OF SOLID STATE CHEM., 178:321-328 (2005), which is incorporated herein by reference in its entirety, describes methods of making mesoporous titania via a combination of sol-gel and solvothermal process at a low temperature.

Mesoporous titania may also be prepared by the hydrolysis of titania precursors in the presence of a surfactant that serves as a scaffold for the porous structure. After precipitation, the resulting powder is treated in hydrothermal or solvothermal conditions to increase the crystallinity of the material. Kim & Kwak, APPLIED CATALYSIS A: GENERAL 323:110-118 (2007), which is incorporated herein by reference in its entirety, describes the hydrothermal synthesis of mesoporous titania with high crystallinity, thermal stability, large surface area, and enhanced photocatalytic activity. After hydrothermally treating the mesoporous titania can be calcined at a temperature above about 300° C. to remove surfactant. The resulting material comprises microscopic particles formed by covalently bonded sub-micron-sized crystalline anatase particles. Since the overall size of these particles is in the microscopic range, the risk of penetrating the stratum corneum is minimized and the presence of small crystalline domains maximizes the absorbance and dispersion of UV radiation. While these particles have very strong absorbance in the UV region, their absorbance quickly decays in the visible region. Therefore, a cosmetic composition comprising these particles can be invisible to the human eye once applied onto the skin, a property desirable in sunscreen formulations.

The cosmetic compositions may include mesoporous titania that has been hydrothermally treated and/or calcined. In one embodiment, the average pore size of the mesoporous titania is from 5.7 to 10.1 nm.

Li et al., MICROPOROUS AND MESOPOROUS MATERIAL, 106:278-283 (2007), which is incorporated herein by reference in its entirety, describes methods of making hierarchical mesoporous grape-like titania with superior photoactivity and recyclability. A bubbling-mediated hydrolysis approach was developed to prepare hierarchically mesoporous titania material with well-defined grape-like morphology in the presence of a triblock copolymer. In one embodiment of the present disclosure, the cosmetic compositions comprise hierarchical mesoporous titania and/or hierarchical mesoporous titania formed by bubbling-mediated hydrolysis. The mesoporous titania of the instant disclosure can take any number of hierarchical shapes.

In addition to the mesoporous titania, the cosmetic compositions may further comprise an inorganic ingredient that provides UV absorption properties such as zinc oxide, titania, iron oxide, zirconium oxide, cerium oxide, and mixtures thereof. Or, the cosmetic compositions may further comprise an organic active ingredient that provides UV absorption properties such as octinoxate, octisalate, homosalate, avobenzone, octocrylene, para-aminobenzoic acid, cinoxate, dioxybenzone, methyl anthranilate, octocrylene, padimate O, ensulizole, sulisobenzone, trolamine salicylate, ecamsule, and mixtures thereof. The cosmetic compositions may optionally comprise a combination of an inorganic active ingredient and an organic active ingredient that provide UV absorption properties.

The cosmetic compositions provide for the mesoporous titania to be combined with a dermatologically acceptable carrier. Suitable dermatologically acceptable carriers can be liquid or solid. Useful liquid carriers include water, alcohols or glycols or water-alcohol/glycol blends, in which the ingredients can be dissolved or dispersed at effective levels, optionally with the aid of non-toxic surfactants. Useful solid carriers include finely divided solids such as talc, clay, microcrystalline cellulose, silica, alumina and the like.

The dermatologically acceptable carrier may be in the form of an emulsion comprising a hydrophilic phase and a hydrophobic phase. The hydrophilic phase will be dispersed in the hydrophobic phase, or vice versa, to form respectively hydrophilic or hydrophobic dispersed and continuous phases, depending on the composition ingredients. The emulsion may be or comprise (e.g., in a triple or other multi-phase emulsion) an oil-in-water emulsion or a water-in-oil emulsion such as a water-in-silicone emulsion. The total level of oil phase components in the compositions is typically from about 0.1% to about 60%, from about 1% to about 30%, or from about 3% to abut 20% and still from about 5% to about 15% by weight of the total composition. When the composition is in the form of an emulsion, the composition may also optionally comprise a surfactant, typically in an amount of from about 0.1% to about 30% or from about 1% to about 20% by weight based upon the total weight of the composition.

The hydrophilic phase can comprise water, or a combination of water and one or more water soluble or dispersible ingredients. Hydrophilic components comprising a substantial amount of water are typical. The composition typically comprises from about 10% to about 95% or from about 30% to 85% by weight of the hydrophilic diluent, based upon the total weight of the composition.

The oil phase may comprise oily materials such as natural or synthetic oils selected from mineral, vegetable, and animal oils, fats and waxes, fatty acid esters, fatty alcohols, fatty acids and mixtures thereof. For example, saturated and unsaturated fatty alcohols such as benzyl alcohol, cetyl alcohol and stearyl alcohol and hydrocarbons such as mineral oils or petrolatum may be included. The oily phase may further comprise oil-soluble skin care additive. Non-limiting examples of oil-soluble skin care additive suitable for use herein include ceramides, cholesterols, fatty acids, vitamin E or its derivatives, oil-soluble vitamin B₃ compounds, or mixtures thereof. Another oil-soluble skin care additive suitable for use herein comprise the oil-soluble vitamin B₃ compounds including “non-vasodilating” esters of nicotinic acid, examples of which include tocopherol nicotinate. As used herein, the term “non-vasodilating” means that the ester does not commonly yield a visible flushing response after application to the skin in the compositions of interest.

Suitable oily materials that are liquid at room temperature, often referred to as oils, include hydrocarbon-based oils of animal origin such as perhydrosqualene; hydrocarbon-based plant oils such as liquid triglycerides of fatty acids of 4 to 10 carbon atoms, for instance, heptanoic or octanoic acid triglycerides, or oils such as sunflower oil, corn oil, soybean oil, grapeseed oil, castor oil, avocado oil, caprylic/capric acid triglycerides, jojoba oil; linear or branched hydrocarbons of mineral or synthetic origin such as liquid paraffins and derivatives thereof, petroleum jelly; synthetic esters and ethers, in particular esters of fatty alcohols, namely; for example, isopropyl myristate, 2-ethylhexyl palmitate, 2-octyldodecyl stearate, isostearyl isostearate; hydroxylated esters such as isostearyl lactate, octyl hydroxystearate, octyldodecyl hydroxystearate, heptanoates, octanoates and decanoates of fatty alcohols; polyol esters such as propylene glycol dioctanoate, neopentyl glycol diheptanoate, diethylene glycol diisononanoate, and pentaerythritol esters; fatty alcohols containing from 12 to 26 carbon atoms such as octyldodecanol, 2-butyloctanol, 2-hexyldecanol, 2-undecylpentadecanol, oleyl alcohol; partially hydrocarbon-based fluoro oils and/or fluorosilicone oils; silicone oils such as volatile or non-volatile, linear or cyclic polydimethylsiloxanes (PDMS) that are liquid or semisolid at room temperature such as cyclomethicones and dimethicones, optionally comprising a phenyl group, for instance phenyl trimethicones, siloxanes, and mixtures thereof These oils are usually present in an amount of about 0.1% to about 90% or from about 1% to about 80% by weight of the oil phase, based upon the total weight of the composition.

The oil phase of the composition may also comprise one or more cosmetically acceptable organic solvents. These solvents are present in an amount of about 0.5% to about 60% or about 1% to about 30% by weight based on the total weight of the composition, and may be selected from the group consisting of lipophilic organic solvents, amphiphilic organic solvents and mixtures thereof Suitable solvents which may be used in the composition include acetic acid esters such as methyl, ethyl, butyl, amyl or 2-methoxyethyl acetate; isopropyl acetate; hydrocarbons such as toluene, xylene, p-xylene, hexane or heptane; ethers containing at least 3 carbon atoms, and mixtures thereof.

The oil phase of the composition may comprise one or more waxes, gums, or mixtures thereof The waxes include hydrocarbon-based waxes, fluoro waxes and/or silicone waxes and can be of plant, mineral, animal and/or synthetic origin. In particular, the waxes have a melting point of greater than 25° C., often greater than 45° C. The compositions may contain from about 0.1% to about 20% by weight of waxes, based upon the total weight of the composition.

The cosmetic composition may further comprise a silicone phase. The silicone phase can comprise one or more silicone components such as silicone fluids, gums, and mixtures thereof. The silicone phase generally comprises from about 0.1% to about 20%, from about 0.2% to about 10%, or from about 0.3% to about 5% by weight of the composition, based upon the total weight of the composition.

In one embodiment, the composition includes an aqueous phase which contains water or a mixture of water and at least one hydrophilic organic solvent in particular an alcohol, especially a linear or branched lower monoalcohol containing from 2 to 5 carbon atoms, e.g., ethanol or propanol; a polyol, e.g., propylene glycol, sorbitol, glycerol; diglycerol, panthenol, or polyethylene glycol, and mixtures thereof. This aqueous phase may represent from about 0.5% to about 99.99% by weight, based upon the total weight of the composition.

The cosmetic composition may also be utilized with colorants, pigments, or dyes to alter the desired optical properties of the composition or to produce a desired blurring effect, enhanced diffused transmittance, and optimal reflection. Examples of suitable pigments include but are not limited to titanium oxide, zirconium oxide, cerium oxide, zinc oxide, iron oxide, chromium oxide, ferric blue, barium, strontium, calcium, and carbon black.

The cosmetic compositions may also comprise a thickening agent. The thickening agent may be in an amount from about 0.1% to about 5%, from about 0.1% to about 3%, or from about 0.25% to about 2% by weight, based upon the total weight of the composition. Non-limiting examples of thickening agents suitable for use herein include cross-linked acrylate copolymers, hydroxyalkylacrylate copolymers, polyacrylamide polymers, natural gum thickeners, or mixtures thereof. The composition may also comprise a thickening polymer such as an amphiphilic polyurethane, a polyacrylic homopolymer or copolymer, a polyester, or a hydrocarbon-based resin. Other non-limiting polymers include, homopolymers or copolymers of: vinyl esters of an aliphatic aid having 1 to 18 carbon atoms, such as vinyl acetate; acrylic acid esters and methacrylic acid esters of an alcohol having 1 to 18 carbon atoms, such as methyl acrylate, ethyl acrylate, butyl acrylate, 2-ethyhexyl acrylate, methyl methacrylate, ethyl methacrylate and butyl methacrylate; and mono and di-ethylenically unsaturated hydrocarbons, including ethylene iso-butylene, styrene and aliphatic dunes, including butadiene, isoprene and chloroprene.

The cosmetic compositions can optionally include an emollient. An emollient provides the functional benefits of enhancing skin smoothness, reducing the appearance of fine lines and coarse wrinkles, and moisturizing. Non-limiting examples include isopropyl myristate, petrolatum, isopropyl lanolate, silicones (e.g., methicone, dimethicone), oils, mineral oils, fatty acid esters, or any mixtures thereof The emollient is typically present from about 0.1% to about 50% by weight, based upon the total weight of the composition.

### EXAMPLE 1

### Synthesis and Characterization

Mesoporous titania samples were synthesized using a soft template to produce the pores and a hydrothermal treatment to crystallize the material. This procedure is described in detail by Kim and Kwak (D. S. Kim, S-Y Kwak, APPL CATAL A, 323:110-118 (2007). The synthesis was repeated at least 10 times to ensure reproducibility and to produce enough material for further testing. Additional synthesis methods to produce different particle/pore sizes using other templates have been reported in the literature.

The average particle size of the mesoporous titania, determined by electron microscopy images, was about 1.26 microns (standard deviation 0.24 micron, n>170). Granulometry analysis using light scattering and dynamic light scattering showed that 99% of the particles are larger than 0.5 micron; the mode size determined by this method was 1.495 microns which is within one standard deviation from the average size measured by electron microscopy. The specific surface area and pore size was determined from nitrogen adsorption-desorption isotherms. The values were calculated using the Brunauer-Emmett-Teller (BET) (surface area) and Barret-Joyner-Halenda (BJH) (pore size) formulae. Detailed descriptions of the methods can be found in the literature (S. Brunauer, P. H. Emmett and E. Teller, J. AM. CHEM. SOC. 60:309 (1938) 309 and E. Barret, L. Joyner and P. Halenda, J. AM. CHEM. SOC., 73:373-380 (1951)). The values obtained were BET surface area (m³/g)=256.9837 and BJH average pore diameter (nm)=3.0746. Micro-XRD analysis was performed on the Mesoporous titania, and showed the presence of the anatase phase only. The size of the crystallites that form the particles was determined from electron microscopy images of samples that had been embedded in resin and cross sectioned. The average crystallite size was 37.6 nm (standard deviation 8.3 nm).

### EXAMPLE 2

A particle integrity test (particle robustness) was carried out by applying a strong shearing force to the particles. The test entailed dispersing the particles in a test liquid (water and ethanol were used), ultrasonicating the dispersions for 30 minutes, and taking electron microscopy images of a drop of the dispersion deposited on a Cu grid. The test was repeated three times and in all cases, the mesoporous titania particles retained their shape and size, i.e. no release of small particles was found.

In-vitro irritation testing was performed by applying a test suspension of mesoporous titania to EPISKIN®. Irritation and inflammation are indicated by an increase of cytokines (IL-8 and IL-1α) compared to baseline. Concentrations of IL-8 and IL-1α after 24 hr were measured using standard methods. The test suspension (mesoporous titania+glycerin/water as vehicle) was prepared and applied as recommended by the European Cosmetic, Toiletry and Perfumery Association (COLIPA). The test was run using an untreated EPISKIN® section as baseline, one control treated with the vehicle only, and one sample treated with the mesoporous titania -containing suspension. Measurement of cytokine expression (IL-8 and IL-1α) showed no significant differences between the cytokines concentrations in the mesoporous titania-treated sample versus baseline and control. This shows that mesoporous titania does not produce skin irritation or inflammation.

### EXAMPLE 3

### Optical Properties: Efficiency as UV filter vs Cosmeticity

For cosmetic applications it is desired that a material has high UV absorbance with low absorbance and scattering in the visible part of the spectrum. The overall optical benefit of such material can be measured as the ratio of UV absorption/visible scattering. Model data for UV absorption was measured on mesoporous titania dispersions and compared to the absorption of non-porous titanium oxide particles of similar size (commercially available from Aldrich). Light absorption and scattering determined in the 250-700 nm range. The prototype mesoporous titania showed its strongest absorption in the 250-320 nm, which includes the harmful UVB range—the absorption decayed after 350 nm. The absorption of commercial titania was 50% lower than mesoporous titania at 250 nm wavelength and decayed after 370 nm. This result indicates that the mesoporous titania particles may effectively filter UVB radiation from reaching the skin.

The optical properties in the visible region were also analyzed for both samples. Absorption in the visible region produces a white residue on the skin which is undesirable in many cosmetic applications. In the visible region, mesoporous titania showed much weaker absorption than the commercial titanium oxide. Therefore, the mesoporous titania has a better UV absorption/visible scattering ratio (1.7 for mesoporous titania vs 0.5 for non-porous commercial titania).

The foregoing description illustrates and describes the present disclosure. Additionally, the disclosure shows and describes only the preferred embodiments of the disclosure, but, as mentioned above, it is to be understood that it is capable of changes or modifications within the scope of the concept as expressed herein, commensurate with the above teachings and/or skill or knowledge of the relevant art. The embodiments described hereinabove are further intended to explain best modes known of practicing the invention and to enable others skilled in the art to utilize the disclosure in such, or other, embodiments and with the various modification required by the particular applications or uses disclosed herein. Accordingly, the description is not intended to limit the invention to the form disclosed herein. Also, it is intended that the appended claims be construed to include alternative embodiments.

All publications, patents and patent applications cited in this specification are herein incorporated by reference, and for any and all purposes, as if each individual publication, patent or patent application were specifically and individually indicated to be incorporated by reference.

What is claimed:

1. An ultra-violet light absorbing cosmetic composition comprising mesoporous titania and a dermatologically acceptable carrier.

2. The cosmetic composition of claim 1, wherein the cosmetic composition absorbs ultraviolet B light from 280 nm to 320 nm.

3. The cosmetic composition of claim 1, wherein the mesoporous titania has a particle size of from about 0.3 microns to about 300 microns.

4. The cosmetic composition of claim 3, wherein the mesoporous titania has a particle size of from about 0.5 microns to about 50 microns.

5. The cosmetic composition of claim 1, wherein the mesoporous titania has a pore size of from about 2 nm to about 100 nm.

6. The cosmetic composition of claim 1 wherein the mesoporous titania has a pore size of from about 2 nm to about 50 nm.

7. The cosmetic composition of claim 1, wherein the mesoporous titania has a crystallite/wall size of from about 2 nm to about 200 nm.

8. The cosmetic composition of claim 7, wherein the mesoporous titania has a crystallite/wall size of from about 5 nm to about 100 nm.

9. The cosmetic composition of claim 1 further comprising an organic ingredient that provides UV absorption selected from the group consisting of octinoxate, octisalate, homosalate, avobenzone, octocrylene, para-aminobenzoic acid, cinoxate, dioxybenzone, methyl anthranilate, octocrylene, padimate O, ensulizole, sulisobenzone, trolamine salicylate, ecamsule, and mixtures thereof.

10. The cosmetic composition of claim 1, wherein the cosmetic composition is formulated as a sunscreen, lotion, cream, gel, spray, powder, foundation, lipstick, shampoo, and/or conditioner.

11. A method of preventing or reducing ultraviolet light damage to the body comprising applying to the body a cosmetic composition of claim 1.

12. The method of claim 11, wherein the cosmetic composition absorbs ultraviolet B light from 280 nm to 320 nm.

13. The method of claim 11, wherein the mesoporous titania has a particle size of from about 0.3 microns to about 300 microns.

14. The method of claim 13, wherein the mesoporous titania has a particle size of from about 0.5 microns to about 50 microns.

15. The method of claim 11, wherein the mesoporous titania has a pore size of from about 2 nm to about 100 nm.

16. The method of claim 15, wherein the mesoporous titania has a pore size of from about 2 nm to about 50 nm.

17. The method of claim 11, wherein the mesoporous titania has a crystallite/wall size of from about 2 nm to about 200 nm.

18. The method of claim 17, wherein the mesoporous titania has a crystallite/wall size of from about 5 nm to about 100 nm.

19. The method of claim 11, wherein the cosmetic composition further comprising an organic ingredient that provides UV absorption selected from the group consisting of octinoxate, octisalate, homosalate, avobenzone, octocrylene, para-aminobenzoic acid, cinoxate, dioxybenzone, methyl anthranilate, octocrylene, padimate O, ensulizole, sulisobenzone, trolamine salicylate, ecamsule, and mixtures thereof.

20. The method of claim 11, wherein the cosmetic composition is formulated as a sunscreen, lotion, cream, gel, spray, powder, foundation, lipstick, shampoo, and/or conditioner.
